SEQUENCE LISTING (1 sequence)

SEQ ID NO: 1 - protein, 6 aa, Artificial Sequence
  Synthetic Construct
GRGDSP

(19) United States
(12) Patent Application Publication
(10) Pub. No.: US 2009/0081276 A1
(43) Pub. Date: Mar. 26, 2009

(54) BIORESORBABLE IMPLANT COMPOSITION

(76) Inventors: EBEN ALSBERG, Cleveland, OH (US); ARNOLD I. CAPLAN, Cleveland Heights, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/191,034

(22) Filed: Aug. 13, 2008

Related U.S. Application Data
(60) Provisional application No. 60/955,523, filed on Aug. 13, 2007.

(51) Int. Cl. A61F 2/02; A61K 35/00; A61K 47/36; A61K 38/18; A61K 31/7088; A61K 31/7105

(52) U.S. Cl. 424/426; 424/93.7; 514/779; 514/12; 514/44

(57) ABSTRACT

A bioresorbable implant composition includes a polymeric macro- or micro-scaffold and first and second bioactive agents respectively incorporated on or within the polymeric macro- or micro-scaffold. The first and second bioactive agents modulate a different function and/or characteristic of a cell.

### RELATED APPLICATION

This application claims priority from U.S. Provisional Application No. 60/955,523, filed Aug. 13, 2007, the subject matter, which is incorporated herein by reference.

### TECHNICAL FIELD

The present invention generally relates to tissue engineering, and more particularly relates to a bioresorbable implant composition and method for promoting tissue growth (e.g., bone and/or cartilage) in a subject.

### BACKGROUND OF THE INVENTION

Articular cartilage has inadequate intrinsic ability to repair itself when damaged through injury or degenerative joint disease. Current methods to address this problem have been met with limited success. Subchondral bone marrow stimulation techniques, such as abrasion arthroplasty, subchondral drilling, and microfracture often result in the formation of fibrocartilage. Osteochondral, periosteal, and perichondral autografts utilized to treat cartilage defects can be complicated by problems associated with donor-site morbidity, limited available tissue, and surface incongruity. Allografts similarly suffer from limited supply and possible disease transmission or immunorejection.

Autologous mesenchymal stem cells (MSCs) obtained from the bone marrow of patients are a promising potential cell source for cartilage regeneration. One of the primary obstacles in using such an approach, however, is controlling the differentiation of MSCs into mature chondrocytes once they are implanted. The differentiation of MSCs into a chondrogenic phenotype during the natural chondrogenesis process occurs in a sequence of events regulated by the temporal presentation of growth, differentiation, and transcription factors.

### SUMMARY OF THE INVENTION

The present invention generally relates to tissue engineering, and more particularly relates to a bioresorbable implant composition and method for promoting tissue growth (e.g., bone and/or cartilage) in a subject.

According to one aspect of the present invention, a bioresorbable implant composition comprises a polymeric macro- or micro-scaffold and first and second bioactive agents incorporated respectively on or within the polymeric macro- or micro-scaffold. The first and second bioactive agents modulate a different function and/or characteristic of a cell.

According to another aspect of the present invention, a bioresorbable implant composition comprises a polymeric macro- or micro-scaffold and at least one bioactive agent incorporated on or within the polymeric macro- or micro-scaffold. The at least one calcium phosphate nanoparticle modulates a function and/or characteristic of a cell.

According to another aspect of the present invention, a bioresorbable implant composition comprises a polymeric macro- or micro-scaffold and at least one interfering RNA molecule incorporated on or within the polymeric macro- or micro-scaffold. The at least interfering RNA molecule modulates a function and/or characteristic of a cell.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a flow diagram illustrating a method for forming a bioresorbable implant composition according to an aspect of the invention;

FIG. 2 is a flow diagram illustrating a method for promoting tissue growth in a subject according to another aspect of the present invention;

FIGS. 3A-B are transmission electron microscopy (TEM) photomicrographs of calcium phosphate (CaP)-DNA coat nanoparticles (NPs) (FIG. 3A) and CaP-DNA core-bovine serum albumin (BSA) (FIG. 3B) at t=0 weeks;

FIG. 4 is a graph comparing the size stability of CaP-DNA NPs over two weeks as determined by analysis of TEM images;

FIG. 5 is a photomicrograph of MC3T3-E1 cells transfected by CaP core-DNA coat NPs (X-Gal staining);

FIG. 6 illustrate plots comparing release profiles of CaP-DNA NPs and naked DNA from alginate hydrogels over time;

FIGS. 7A-B are a series of histology slides showing implants composed of MC3T3-E1 cells and CaP core-DNA coat NPs in the alginate after 6 weeks post-injection in mice. FIG. 7A is done with H&E staining, and FIG. 7B is done with Goldner's Trichome staining;

FIG. 8 illustrates plots comparing release profiles of BSA from PLGA microspheres prepared according to Example 9 (below) (2A=20 KDa; 6A=60 KDa);

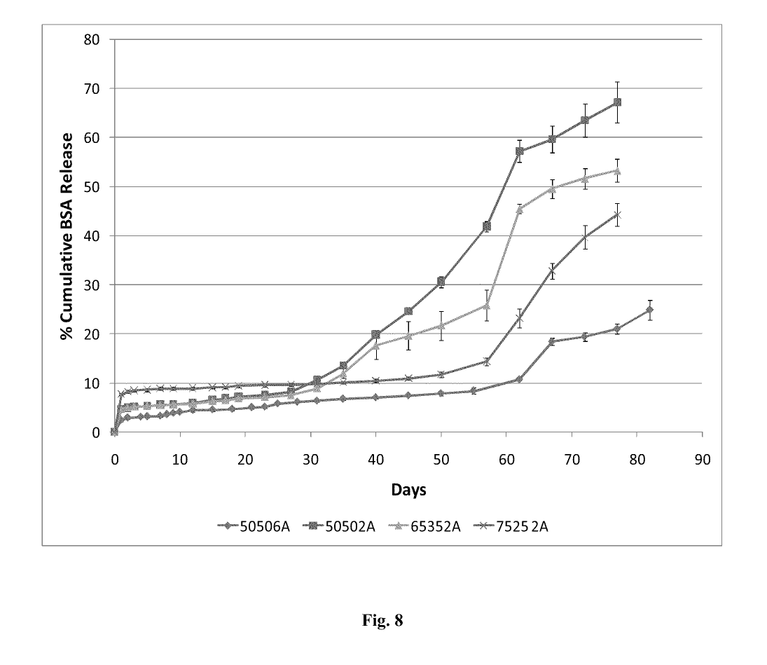

FIG. 9 illustrates plots comparing release profiles of BSA from PLGA microspheres prepared according to Example 10 (below) (2A=20 KDa; 6A=60 KDa);

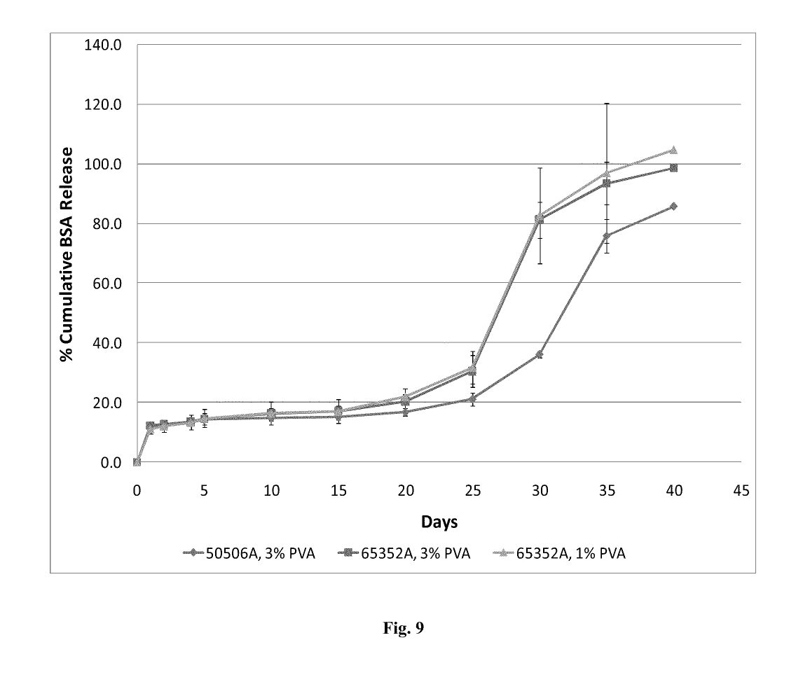

FIG. 10 illustrates plots comparing release profiles of BSA from PLGA microspheres prepared according to Example 10 (below) (2A=20 KDa; 6A=60 KDa);

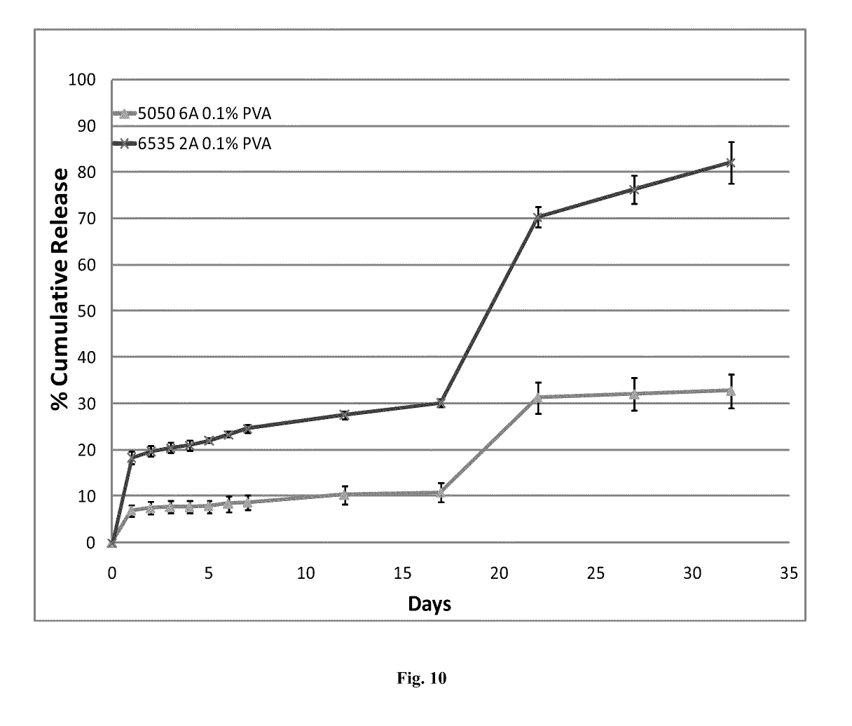

FIG. 11 is a graph comparing knockdown of pDNA encoding shRNA for deGFP in HEK293 cells plated on collagen-coated plates;

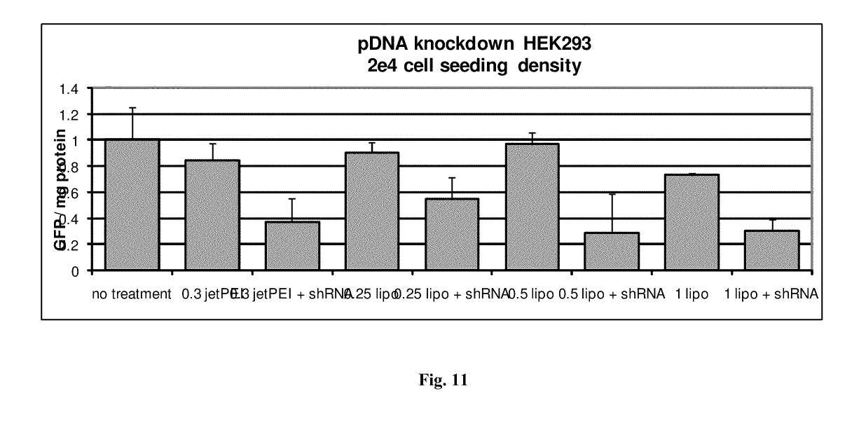

FIG. 12 is a series of phase contrast and fluorescent micrographs showing HEK293 deGFP knockdown;

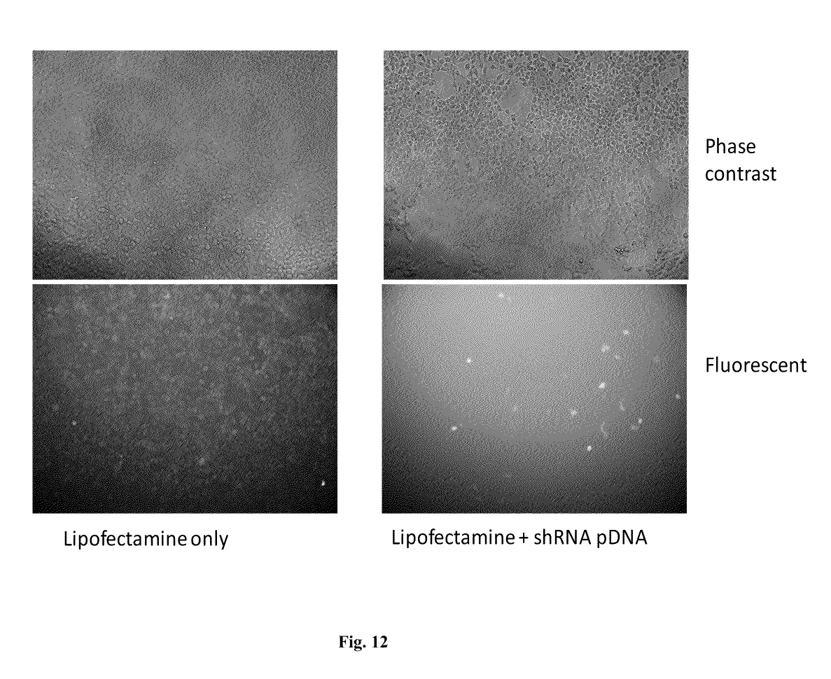

FIG. 13 are graphs comparing deGFP knockdown in HEK293 cells with siRNA and interferin (upper) or lipofectamine (lower);

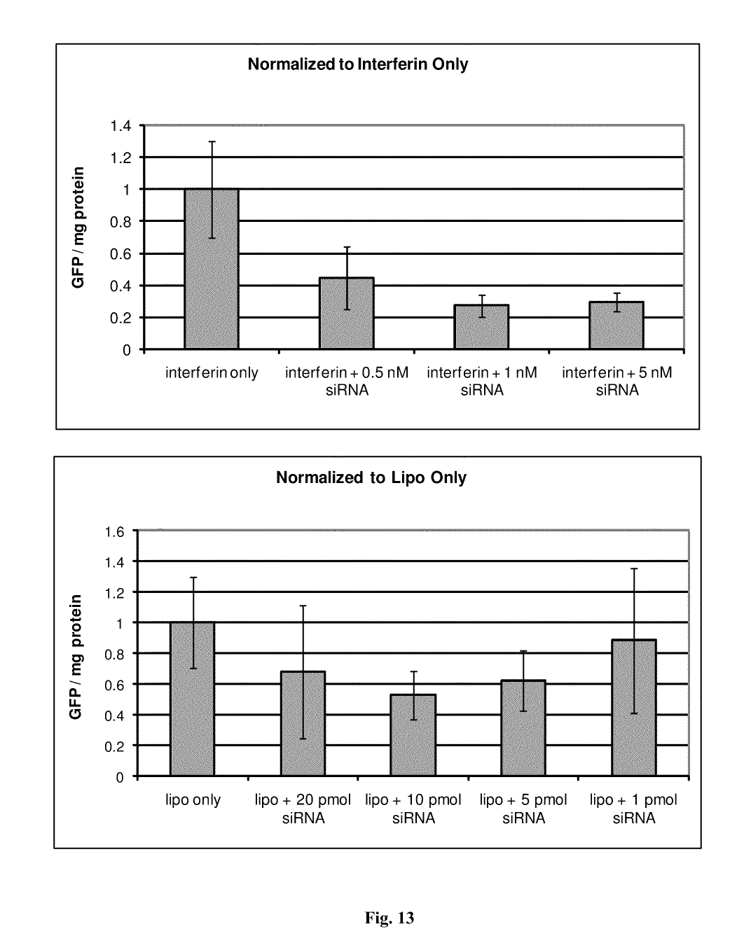

FIG. 14 illustrates plots comparing DNA release from different PLGA scaffolds in DMEM without calcium cross-linking (N=1 for all conditions);

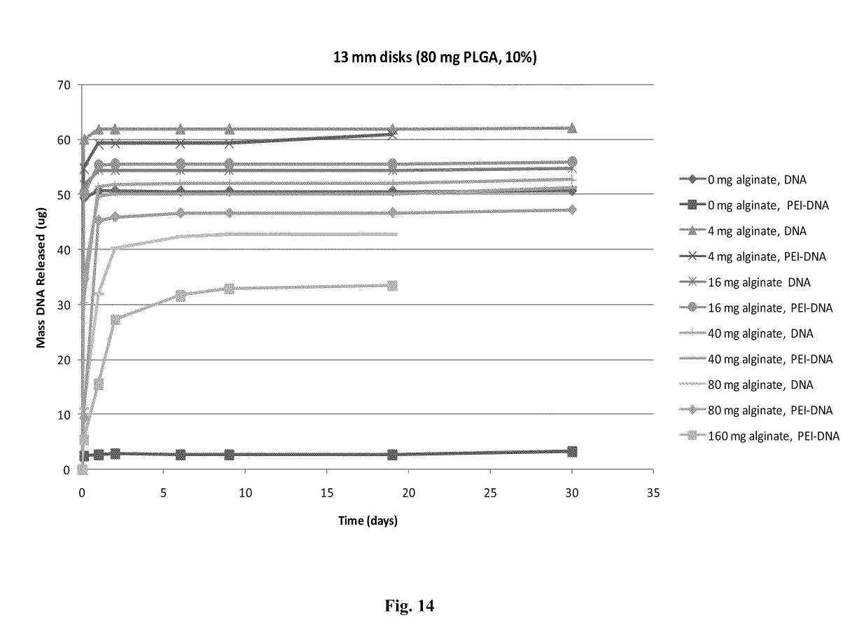

FIG. 15 illustrate plots comparing DNA release from different PLGA scaffolds (10% PLGA scaffold (80 mg) soaked in DMEM with 0.1 M CaCl₂ for 3 hours) (N=1 for all conditions);

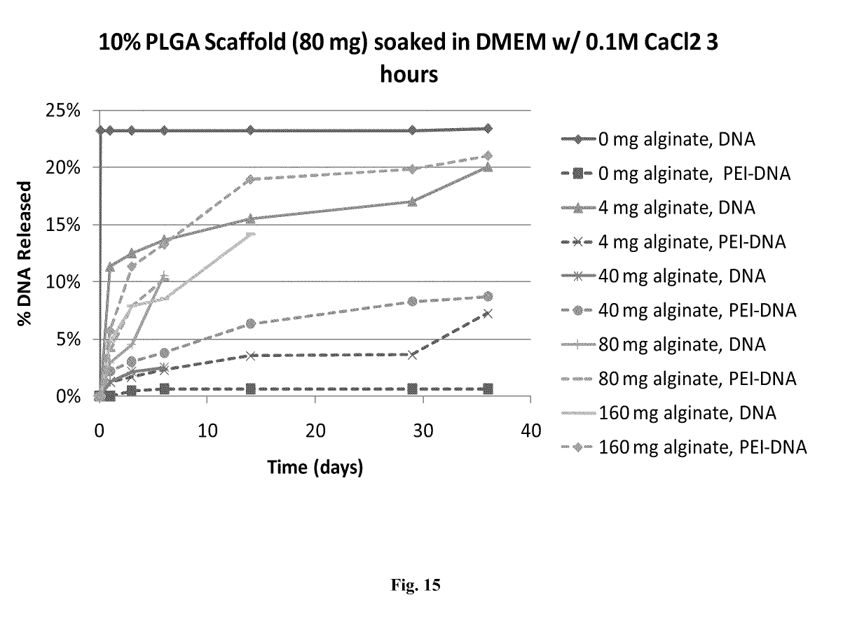

FIG. 16 illustrate plots comparing DNA release from different PLGA scaffolds (10% PLGA scaffold (80 mg) with 220 mg CaCl₂) (N=1 for all conditions);

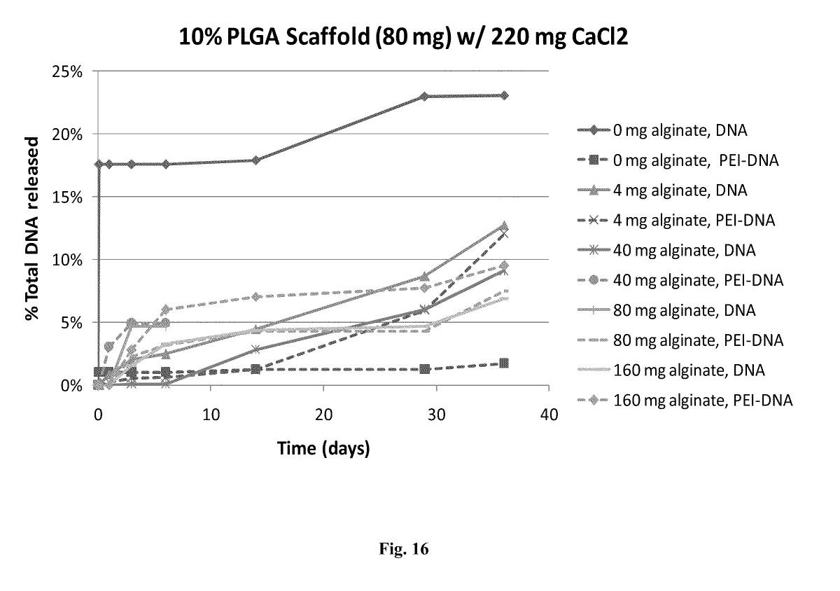

FIG. 17 illustrate plots comparing DNA release from different PLGA scaffolds cross-linked with CaCl₂ for 1 minute (N=1 for all conditions); and

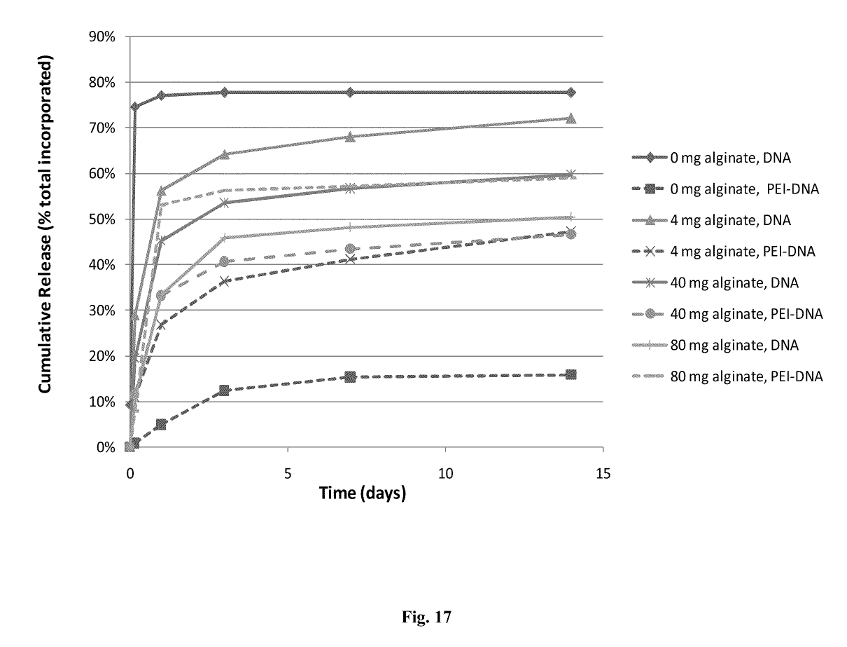

FIG. 18 illustrate plots comparing DNA release from different PLGA scaffolds soaked in DMEM-HG and cross-linked with CaCl₂ for 1 minute (N=4 for all conditions).

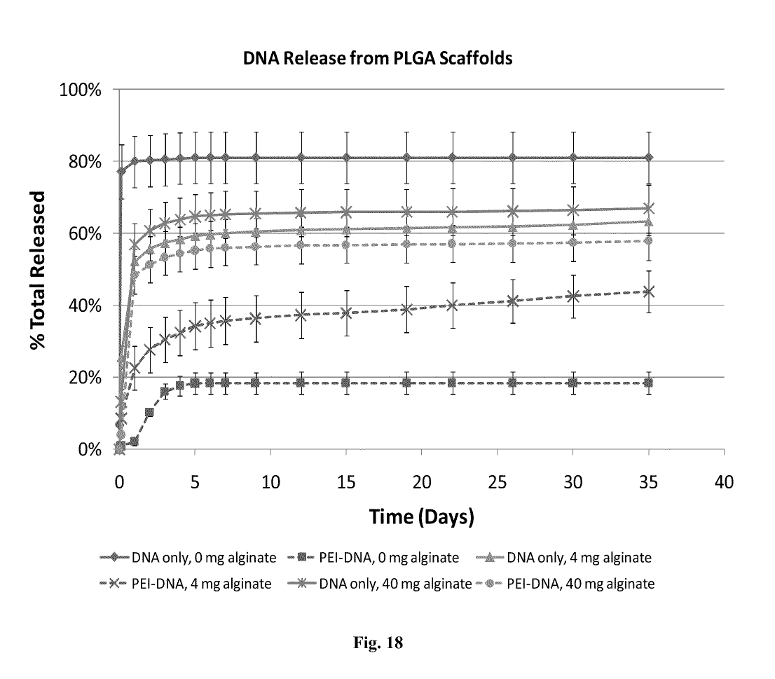

### DETAILED DESCRIPTION

The present invention generally relates to tissue engineering, and more particularly relates to a bioresorbable implant composition and method for promoting tissue growth (e.g., bone and/or cartilage) in a subject. The present invention provides a bioresorbable implant composition that is capable of controlled release of bioactive agents, such as polypeptides and polynucleotides, to, for example, mimic the temporal sequence of growth factor and/or cytokine release during tissue growth or healing. The bioresorbable implant composition can be injected or implanted in a minimally invasive fashion at a tissue defect (e.g., a cartilage or bone defect) to repair damaged tissue (e.g., bone and/or cartilage) and/or treat diseases (e.g., cancer) in a subject.

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the present invention.

In the context of the present invention, the term “bioactive agent” can refer to any agent capable of promoting tissue formation, destruction, and/or targeting a specific disease state (e.g., cancer). Examples of bioactive agents can include, but are not limited to, chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-I, IGF-II) and transforming growth factors (e.g., TGF-β I-III)), parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (e.g., MP52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

As used herein, the term “bioresorbable” can refer to the ability of a material to be fully resorbed in vivo. “Full” can mean that no significant extracellular fragments remain. The resorption process can involve elimination of the original implant material(s) through the action of body fluids, enzymes, cells, and the like.

As used herein, the term “carrier material” can refer to a material capable of transporting, releasing, and/or complexing at least one bioactive agent.

As used herein, the term “function and/or characteristic” can refer to the modulation or proliferation of at least one progenitor cell, the modulation of the state of differentiation of at least one progenitor cell, and/or the induction of a pathway in at least one progenitor cell, which directs the cell to differentiate along a desired pathway, e.g., leading to a desired cell phenotype, cell migration, angiogenesis, apoptosis, etc.

As used herein, the term “polymeric macro- or micro-scaffold” can refer to a biodegradable or non-biodegradable biocompatible material, which serves as a material or macro- or micro-scaffold for incorporation of at least one carrier material, at least one cell, and/or bioactive agent of the present invention.

As used herein, the term “polynucleotide” can refer to oligonucleotides, nucleotides, or to a fragment of any of these, to DNA or RNA (e.g., mRNA, rRNA, siRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, ribonucleoproteins (e.g., iRNPs). The term can also encompass nucleic acids, i.e., oligonucleotides, containing known analogues of natural nucleotides. Additionally, the term can encompass nucleic acid-like structures with synthetic backbones.

As used herein, the term “polypeptide” can refer to an oligopeptide, peptide, polypeptide, or protein sequence, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. Additionally, the term “polypeptide” can include peptides and polypeptide fragments, motifs and the like, glycosylated polypeptides, and all “mimetic” and “peptidomimetic” polypeptide forms.

As used herein, the term “cell” can refer to any progenitor cell, such as totipotent stem cells, pluripotent stem cells, and multipotent stem cells, as well as any of their lineage descendant cells, including more differentiated cells. The terms “stem cell” and “progenitor cell” are used interchangeably herein. The cells can derive from embryonic, fetal, or adult tissues. Exemplary progenitor cells can be selected from, but not restricted to, totipotent stem cells, pluripotent stem cells, multipotent stem cells, mesenchymal stem cells (MSCs), hematopoietic stem cells, neuronal stem cells, hematopoietic stem cells, pancreatic stem cells, cardiac stem cells, embryonic stem cells, embryonic germ cells, neural crest stem cells, kidney stem cells, hepatic stem cells, lung stem cells, hemangioblast cells, and endothelial progenitor cells. Additional exemplary progenitor cells are selected from, but not restricted to, de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

As used herein, the term “subject” can refer to any animal, including, but not limited to, humans and non-human animals (e.g., rodents, arthropods, insects, fish (e.g., zebrafish), non-human primates, ovines, bovines, ruminants, lagomorphs, porcines, caprines, equines, canines, felines, aves, etc.), which is to be the recipient of a particular treatment. Typically, the terms “patient” and “subject” are used interchangeably herein in reference to a human subject.

As used herein, the term “tissue” can refer to an aggregate of cells having substantially the same function and/or form in a multicellular organism. “Tissue” is typically an aggregate of cells of the same origin, but may be an aggregate of cells of different origins. The cells can have the substantially same or substantially different function, and may be of the same or different type. “Tissue” can include, but is not limited to, an organ, a part of an organ, bone, cartilage, skin, neuron, axon, blood vessel, cornea, muscle, fascia, brain, prostate, breast, endometrium, lung, pancreas, small intestine, blood, liver, testes, ovaries, cervix, colon, stomach, esophagus, spleen, lymph node, bone marrow, kidney, peripheral blood, embryonic, or ascite tissue.

As used herein, the terms “inhibit,” “silencing,” and “attenuating” can refer to a measurable reduction in expression of a target mRNA (or the corresponding polypeptide or protein) as compared with the expression of the target mRNA (or the corresponding polypeptide or protein) in the absence of an interfering RNA molecule of the present invention. The reduction in expression of the target mRNA (or the corresponding polypeptide or protein) is commonly referred to as “knock-down” and is reported relative to levels present following administration or expression of a non-targeting control RNA.

One aspect of the present invention relates to a bioresorbable implant that includes a polymeric macro- or micro-scaffold and at least one bioactive agent incorporated on or within the polymeric macro- or micro-scaffold. The polymeric macro- or micro-scaffold serves as a substrate for the incorporation and/or attachment of at least one bioactive agent. The polymeric macro- or micro-scaffold may be in the form of a membrane, sponge, gel, solid scaffold, woven or unwoven mesh, or any other desirable configuration.

The polymeric macro- or micro-scaffold can be injectable or implantable and be formed from a natural material, a synthetic material, or a combination thereof. The material used to form the polymeric macro- or micro-scaffold can be a biodegradable polymer so that no, or very little, of the macro- or micro-scaffold remains after new tissue (e.g., cartilage and/or bone) has formed. The polymeric macro- or micro-scaffold may also comprise an inorganic or organic composite. Examples of materials that can be used to form the polymeric macro- or micro-scaffold include chitosan, poly(ethylene oxide), poly (lactic acid), poly(acrylic acid), poly(vinyl alcohol), poly(urethane), poly(N-isopropyl acrylamide), poly(vinyl pyrrolidone) (PVP), poly (methacrylic acid), poly(p-styrene carboxylic acid), poly(p-styrenesulfonic acid), poly(vinylsulfonicacid), poly(ethyleneimine), poly(vinylamine), poly(anhydride), poly(L-lysine), poly(L-glutamic acid), poly(gamma-glutamic acid), poly(carprolactone), polylactide, poly(ethylene), poly(propylene), poly(glycolide), poly(lactide-co-glycolide), poly(amide), poly(hydroxylacid), poly(sulfone), poly(amine), poly(saccharide), poly(HEMA), poly(anhydride), collagen, gelatin, glycosaminoglycans (GAG), poly (hyaluronic acid), poly(sodium alginate), alginate, hyaluronan, agarose, polyhydroxybutyrate (PHB), and combinations thereof.

It will be appreciated that the polymeric macro- or micro-scaffold can have any desired configuration, structure, or density. For example, the polymer concentration, molecular weight, physical or chemical properties, crosslinker type or concentration, solvent concentration, heating temperature, reaction time, and other parameters can be varied to create a polymeric macro- or micro-scaffold with a desired physical and/or biochemical characteristic(s).

In one example, the polymeric macro- or micro-scaffold may be formed into a sponge-like structure with controlled porosity. In another example, the polymeric macro- or micro-scaffold may be formed into a membrane or sheet, which can then be wrapped around or otherwise shaped to a tissue defect (e.g., bone and/or cartilage). The polymeric macro- or micro-scaffold may be configured as a gel, mesh, plate, screw, plug, rod, microbead or macrobead. The polymer macro- or micro-scaffold can take the form of a non-woven or woven mesh of micro- or nano-fibers fabricated by, for example, electrospinning techniques. Any conceivable shape or form of the polymeric macro- or micro-scaffold is within the scope of the present invention.

Polymers used to form the polymeric macro- or micro-scaffold may be cross-linked with a cross-linking agent in order to enhance the mechanical strength of the macro- or micro-scaffold. Examples of cross-linking agents may include divalent cations, genipin, glutaraldehyde, tri-polyphosphate (TPP), hydroxyapitite (HA), and any other cross-linking agent known to those skilled in the art. Alternatively, a cross-linking agent, such as HA, may be coated onto the surface of an already formed polymeric macro- or micro-scaffold.

Other materials known in the art may also be combined with a polymer to form the polymeric macro- or micro-scaffold. For example, calcium phosphate, TCP, collagen, and/or polymethyl methacrylate may be combined with a polymer to form the polymeric macro- or micro-scaffold. At least about 50% of the polymeric macro- or micro-scaffold may be comprised of calcium phosphate, TCP, HA, collagen, polymethyl methacrylate, and/or a mixture thereof.

In an aspect of the invention, at least one attachment molecule, such as a polypeptide or a small molecule, may be chemically immobilized onto the polymeric macro- or micro-scaffold to facilitate cell attachment. Examples of attachment molecules can include fibronectin or a portion thereof, collagen or a portion thereof, polypeptides or proteins containing the arginine-glycine-aspartate sequence (or other attachment sequence), enzymatically degradable peptide linkages, and/or protein-sequestering peptide sequences.

In an example of the present invention, the polymeric macro- or micro-scaffold can comprise a polypeptide-modified alginate macro- or micro-scaffold. The alginate macro- or micro-scaffold can comprise a non-crosslinked alginate, such as alginate commercially available from FMC BIOPOLYMER (Princeton, N.J.). Another example of an alginate macro- or micro-scaffold for use with the present invention is ALGIMED (Cardio Tech International, Inc., Wilmington, Mass.), a calcium alginate composition commonly used as a wound dressing. The polypeptide can have the amino acid sequence of SEQ ID NO: 1 and be chemically immobilized on a portion of the macro- or micro-scaffold.

The at least one bioactive agent can include polynucleotides and/or polypeptides encoding or comprising, for example, transcription factors, differentiation factors, growth factors, and combinations thereof. The at least one bioactive agent can also include any agent capable of promoting tissue formation (e.g., bone and/or cartilage), destruction, and/or targeting a specific disease state (e.g., cancer). Examples of bioactive agents include chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., EGF), HGF, VEGF, fibroblast growth factors (e.g., bFGF), PDGF, insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), sonic hedgehog, growth differentiation factors (e.g., GDF5, GDF6, GDF8), recombinant human growth factors (e.g., MP-52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, DNA encoding for an shRNA of interest, oligonucleotides, proteoglycans, glycoproteins, and glycosaminoglycans.

In another aspect of the present invention, the bioresorbable implant composition can further include at least one cell dispersed on or within the polymeric macro- or micro-scaffold. The at least one cell can include any progenitor cell, such as a totipotent stem cell, a pluripotent stem cell, or a multipotent stem cell, as well as any of their lineage descendant cells, including more differentiated cells (described above). For example, progenitor cells can comprise CD34⁺ MSCs. The cells can include autologous cells; however, it will be appreciated that xenogeneic, allogeneic, or syngeneic cells may also be used. Where the cells are not autologous, it may be desirable to administer immunosuppressive agents in order to minimize immunorejection. The cells employed may be primary cells, expanded cells, or cell lines, and may be dividing or non-dividing cells. Cells may be expanded ex vivo prior to introduction into the polymeric macro- or micro-scaffold. For example, autologous cells can be expanded in this manner if a sufficient number of viable cells cannot be harvested from the host. Alternatively or additionally, the cells may be pieces of tissue, including tissue that has some internal structure. The cells may be primary tissue explants and preparations thereof, cell lines (including transformed cells), or host cells.

In one example of the present invention, the bioresorbable implant composition can comprise a polymeric macro- or micro-scaffold, at least one cell dispersed within or on the polymeric macro- or micro-scaffold, and at least one carrier material incorporated on or within the polymeric macro- or micro-scaffold. The at least one carrier material can include a material capable of carrying and differentially and/or controllably releasing at least one bioactive agent. Carrier materials can be directly linked to the bioactive agent and/or physically associated with the bioactive agent. Carrier materials can include a variety of known microparticles or nanoparticles including, for example, polymer-based and calcium phosphate-based microparticles and nanoparticles. It will be appreciated that a carrier molecule, such as a positively-charged polymer (e.g., PEI) can be included along with a desired bioactive agent (e.g., a DNA plasmid encoding an siRNA or siRNA molecule).

Polymer-based carrier materials can include a biodegradable polymer capable of controllably and/or differentially releasing at least one bioactive agent. For example, a polymer-based carrier material can be a biodegradable polymer in microparticle form. Microparticles can have a diameter less than 1 mm and typically between 1 and 200 microns. Microparticles can include both microspheres and microcapsules, and may have an approximately spherical geometry and be of fairly uniform size. Microspheres can have a homogeneous composition, and microcapsules can include a core composition (e.g., a bioactive agent) distinct from a surrounding shell. For the purposes of the present invention, the terms “microsphere,” “microparticle,” and “microcapsule” may be used interchangeably.

Microparticles can be made with a variety of biocompatible and biodegradable polymers. Examples of biocompatible, biodegradable polymers are poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide)s, biodegradable polyurethanes, and blends and/or copolymers thereof.

Other examples of materials that may be used to form microparticles can include chitosan, poly(ethylene oxide), poly (lactic acid), poly(acrylic acid), poly(vinyl alcohol), poly(urethane), poly(N-isopropyl acrylamide), poly(vinyl pyrrolidone) (PVP), poly (methacrylic acid), poly(p-styrene carboxylic acid), poly(p-styrenesulfonic acid), poly(vinylsulfonicacid), poly(ethyleneimine), poly(vinylamine), poly(anhydride), poly(L-lysine), poly(L-glutamic acid), poly(gamma-glutamic acid), poly(carprolactone), polylactide, poly(ethylene), poly(propylene), poly(glycolide), poly(lactide-co-glycolide), poly(amide), poly(hydroxylacid), poly(sulfone), poly(amine), poly(saccharide), poly(HEMA), poly(anhydride), collagen, fibrin, gelatin, glycosaminoglycans (GAG), poly (hyaluronic acid), poly(sodium alginate), alginate, hyaluronan, agarose, polyhydroxybutyrate (PHB), copolymers thereof, and blends thereof.

In one example of the present invention, a carrier material can comprise a microparticle made of poly(d,l-lactide-co-glycolide) (PLGA). PLGA degrades when exposed to physiological pH and hydrolyzes to form lactic acid and glycolic acid, which are normal byproducts of cellular metabolism. The disintegration rate of PLGA polymers may vary depending on the polymer molecular weight, ratio of lactide to glycolide monomers in the polymer chain, and stereoregularity of the monomer subunits. For example, mixtures of L and D stereoisomers that disrupt the polymer crystallinity can increase polymer disintegration rates. In addition, it will be appreciated that microspheres may contain blends of two or more biodegradable polymers of different molecular weight and/or monomer ratio.

Carrier materials can alternatively comprise a nanoparticle, such as submicron particles, for controlled release of the bioactive agent. A nanoparticle can have a diameter ranging from about less than 1 nanometer to about 1 micron. Nanoparticles can be created in the same manner as microparticles, except that high-speed mixing or homogenization may be used to reduce the size of the nanoparticle/bioactive agent emulsion(s) to less than about 2 microns. Alternative methods for nanoparticle production are known in the art and may be employed for the present invention.

In another example of the present invention, the bioresorbable implant composition can comprise a polymeric macro- or micro-scaffold, at least one cell dispersed within the polymeric macro- or micro-scaffold, and at least one bioactive agent incorporated on or within at least one calcium phosphate nanoparticle dispersed within the polymeric macro- or micro-scaffold. The at least one calcium phosphate nanoparticle can differentially or controllably release the at least one bioactive agent or be taken up (e.g., via endocytosis) by at least one progenitor cell to modulate the function and/or characteristic of the at least one cell.

The at least one bioactive agent may be at least partially coated on the surface of at least one calcium phosphate nanoparticle. Alternatively, the at least one bioactive agent may be dispersed, incorporated, and/or impregnated within the calcium phosphate nanoparticle. For example, a bioactive agent comprising a DNA plasmid (e.g., a plasmid encoding BMP-2) can be coated onto the surface of the calcium phosphate nanoparticle. Alternatively, a DNA plasmid can be co-precipitated with calcium phosphate to form the calcium phosphate nanoparticle. After forming the calcium phosphate nanoparticles, the nanoparticles can be coated with DNA or protein to prevent nanoparticle aggregation and/or promote cellular uptake. It will be appreciated that one or more of the same or different bioactive agents can be incorporated on or within the at least one calcium phosphate nanoparticle.

Calcium phosphate nanoparticles can have an average particle size of between about 1 nm and about 200 nm. It will be appreciated that smaller or larger calcium phosphate nanoparticles may be used. The calcium phosphate nanoparticles can have a generally spherical morphology and be of a substantially uniform size or, alternatively, may be irregular in morphology.

Calcium phosphate nanoparticles may be complexed with surface modifying agents to provide a threshold surface energy sufficient to bind material (e.g., bioactive agents) to the surface of the nanoparticle without denaturing the material. Non-limiting examples of surface modifying agents can include basic or modified sugars, such as cellobiose, carbohydrates, carbohydrate derivatives, macromolecules with carbohydrate-like components characterized by an abundance of —OH side groups, and polyethylene glycol.

In another example of the present invention, a bioresorbable implant composition can comprise a polymeric macro- or micro-scaffold, at least one cell dispersed within the polymeric macro- or micro-scaffold, and at least one interfering RNA molecule incorporated on or within at least one carrier material dispersed within the polymeric macro- or micro-scaffold. The interfering RNA molecule can include any RNA molecule that is capable of silencing a target mRNA and thereby reducing or inhibiting expression of a polypeptide encoded by the target mRNA. Alternatively, the interfering RNA molecule can include a DNA molecule encoding for a shRNA of interest. For example, the interfering RNA molecule can comprise a short interfering RNA (siRNA) or microRNA molecule capable of silencing a target mRNA that encodes any one or combination of the polypeptides or proteins described above. The at least one carrier material can differentially or controllably release the at least one interfering RNA molecule or be taken up (e.g., via endocytosis) by at least one cell to modulate a function and/or characteristic of the at least one cell.

The at least one interfering RNA molecule may be at least partially coated on the surface of the at least one carrier material or, alternatively, dispersed, incorporated, and/or impregnated within the at least one carrier material. For example, a carrier material comprising a PLGA microparticle can be impregnated with an siRNA molecule capable of targeting an mRNA corresponding to at least a portion of the GNAS gene. Alternatively, a carrier material comprising a PLGA microparticle can be impregnated with an siRNA molecule capable of targeting an mRNA corresponding to VEGF. It will be appreciated that the carrier material can be coated with a polynucleotide and/or polypeptide to prevent or reduce aggregation and/or promote cellular uptake of the carrier material. It will also be appreciated that the carrier material can include the same or different interfering RNA molecules, and that two, three, or even more carrier materials can be included with the same or different interfering RNA molecules in the bioresorbable implant composition.

In another example of the invention, the at least one bioactive agent incorporated on or within the at least one carrier material can comprise first and second bioactive agents respectively incorporated on or within first and second carrier materials. The first and second carrier materials may comprise the same or different materials. Additionally, the first and second bioactive agents may comprise the same or different agents. As described in further detail below, the first and second carrier materials can differentially, sequentially, and/or controllably release the first and second bioactive agents to modulate a different function and/or characteristic of at least one cell. It will be appreciated that the first carrier material can release the first bioactive agent with a different release profile than the release profile of the second bioactive agent from the second carrier material. Additionally, it will be appreciated that the first carrier material can degrade or diffuse before the degradation or diffusion of the second carrier material or allow for an increased rate of release or diffusion of the first bioactive agent compared to the release of the second bioactive agent. The first and second carrier materials may be dispersed uniformly within the polymeric macro- or micro-scaffold or, alternatively, dispersed such that different densities of carrier materials are dispersed within different portions of the polymeric macro- or micro-scaffold.

The present invention takes advantage of the precise temporal sequence of growth factor and cytokine presentation needed to guide progenitor cell (e.g., mesenchymal stem cell or MSC) behavior during tissue growth (e.g., chondrogenesis). Many growth factors play an important role in tissue repair and regeneration. At the initial stages of bone and/or cartilage repair and regeneration, for example, growth factors such as PDGF and TGF-β are involved in regulating MSC proliferation. Cells will typically express TGF-β, PDGF, and fibroblast growth factors as they proliferate. As cells proliferate over time, expression of growth factors responsible for proliferation generally decrease and expression of growth factors responsible for differentiation generally increase. Growth factors, such as BMPs and IGF may be expressed at the differentiation stage to drive the cells toward mature chondrocytes, for example, capable of synthesizing new cartilage tissue.

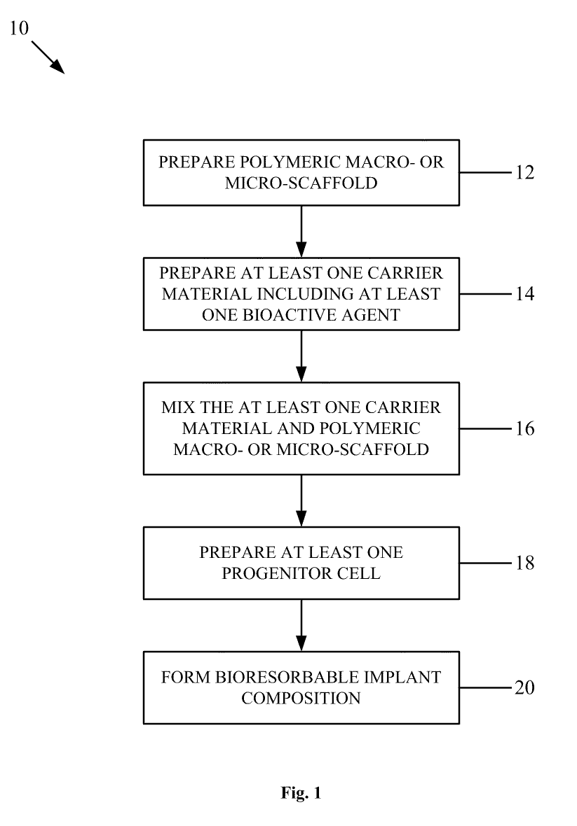

FIG. 1 is a flow diagram illustrating a method 10 for forming a bioresorbable implant composition in accordance with one example of the invention. The method 10 at step 12 includes preparing a polymeric macro- or micro-scaffold. Depending upon the desired use of the bioresorbable implant composition, the polymeric macro- or micro-scaffold can be made of any biodegradable and biocompatible material (described above) capable of serving as a substrate for the incorporation and/or attachment of at least one bioactive agent and at least one cell. In an example of the present invention, the polymeric macro- or micro-scaffold can comprise a polypeptide-modified alginate macro- or micro-scaffold. To form the polymeric macro- or micro-scaffold, a desired quantity of sodium alginate powder can be subjected to gamma irradiation at about 5 MRad. After irradiating the alginate macro- or micro-scaffold, at least one polypeptide having the amino acid sequence of SEQ ID NO: 1 can be synthesized and covalently coupled to the alginate in a manner similar to the method described by Rowley et al., Biomaterials 20(1):45-53 (1999). Next, the alginate macro- or micro-scaffold can be lyophilized until dry, purified by dialysis, subjected to activated charcoal treatment, and then sterilized through a filter, such as a 0.22 μm filter.

In one example, a desired amount of at least one bioactive agent can then be added to the polymeric macro- or micro-scaffold. For example, a desired amount of a bioactive agent, such as about 50 μg to about 400 μg DNA can be added to about a 10 mM HEPES solution (at a pH of about 7.4). The mixture can be incubated at about room temperature for about 30 minutes. Then, about a 2% alginate solution can be added drop-wise while gently vortexing so that the final mass of the alginate is between about 4 mg and about 40 mg. This mixture can be incubated at about room temperature for about 30 minutes. A solution of sucrose in water can be added so that the final sucrose concentration in the mixture is about 1% w/v. This can be added drop-wise with gentle vortexing and then allowed to sit at about room temperature for about 30 minutes. The mixture can then be flash-frozen in liquid nitrogen and subsequently lyophilized until dry (e.g., about 4 days).

After drying, the lyophilized DNA-alginate mixture can be mixed with milled sucrose (e.g., about 250 μm to about 425 μm) and PLGA 50:50 copolymer (e.g., between about 106 μm to about 250 μm). The polymeric macro- or micro-scaffold can be prepared at about 90 wt % (porogen:PLGA) by combining about 720 mg of sucrose and lyophilized DNA-alginate mixture with about 80 mg PLGA. The materials can be mixed together and pressed in about a 13 mm die for about 1 minute at about 3.5 metric tons. The compressed pellet (about 4×13 mm) can then be foamed into a scaffold by placing it in a stainless steel high-pressure vessel and exposing it to dry CO₂ gas at about 800 psi for about 20 hours. The scaffolds can be placed into about 0.1 M CaCl₂ for about 1 minute to crosslink the alginate, and then placed in PBS or media (e.g., DMEM) for about 24 hours to leach the sucrose.

In another example, at least one carrier material that includes at least one bioactive agent can be prepared at step 14. As described above, the at least one carrier material can include a microparticle or nanoparticle of PLGA, calcium phosphate, or a combination thereof. Methods for forming PLGA microparticles are known in the art and generally include combining L and D lactide and glycolide monomers in a desired ratio to impart a particular property, such as disintegration rate to the microparticles. Additionally, methods for making calcium phosphate nanoparticles are known in the art and typically include reacting a soluble calcium salt with a soluble phosphate salt under aseptic conditions. In an example of the method, calcium phosphate nanoparticles coated with DNA, such as a plasmid encoding for BMP-2 can be created by a slight modification of the method described by Sokolova V. V. et al., Biomaterials 27(16):3147-3153 (2006). For instance, approximately equal volumes of about 18.7 mM CaCₗ₂ (at about pH 9) and about 11.23 mM Nₐ₂HP_{O4} (at about pH 9) can be added simultaneously to a tube with a magnetic stir bar. The solution can then be mixed for about 30 seconds and about 200 μg of the DNA added to quench crystallization by coating the crystals.

In another example of the present invention, PLGA microspheres including a bioactive agent (e.g., a protein) can be synthesized by preparing the following solutions: (a) about 5% w/v PLGA in ethyl acetate; (b) an aqueous solution including the protein; (c) a secondary emulsion solution comprising about 5% w/v poly(vinyl alcohol) (PVA, MW 9-10 KDa), and about 7% ethyl acetate in water; and (d) an extraction solution comprising about 0.3% w/v PVA and about 7% ethyl acetate in water. To prepare a primary emulsion, about 100 μl of the protein solution can be added to about 1 ml of the PLGA solution and then sonicated for about 15 seconds at a frequency of about 20 W. This can be done in an ice bath to avoid overheating. Next, about 1 ml of the PVA solution can be added to the primary emulsion and then vortexed at a maximum speed for about 15 seconds. The secondary emulsion can then be poured into about 200 ml of the extraction solution and stirred continuously for about 3 hours. The extraction solution with the microspheres can be filtered through a vacuum. The microspheres can then be rinsed off of the filter with water, poured into about a 50 ml conical tube through about a 70 μm cell strainer, and centrifuged for about 10 minutes at about 7000 rpm. The supernatant can be discarded and the microspheres resuspended in water. This step can be performed two or more times. The collected microspheres can then be flash frozen in liquid nitrogen for about 5 minutes, followed by lyophilization.

In an alternative example, PLGA microspheres including a bioactive agent (e.g., a protein) can be synthesized by preparing the following solutions: (a) about 5% w/v PLGA in ethyl acetate; (b) an aqueous protein solution; and (c) about 0.1% to about 3% PVA w/v (MW 30-70 KDa) for the secondary emulsion. To prepare a primary emulsion, about 1 ml of the aqueous protein solution can be added to about 10 ml of the PLGA solution and then sonicated for about 15 seconds at a frequency of about 20 W. This can be done in an ice bath to avoid overheating. Next, the primary emulsion can be poured into about 200 ml of the PVA solution and then homogenized at about 10,000 rpm for about 1 minute using an ice bath to avoid overheating. The secondary emulsion can be stirred continuously for about 3 hours, followed by filtering of the extraction solution (with the microspheres) through a vacuum filter. The microspheres can then be rinsed off of the filter with water, poured into about a 50 ml conical tube through about a 70 μm cell strainer, and centrifuged for about 10 minutes at about 7,000 rpm. The supernatant can be discarded and the microspheres resuspended in water. This step can be repeated two or more times. The collected microspheres can then be flash frozen in liquid nitrogen for about 5 minutes, followed by lyophilization.

In another example, a carrier material including plasmid DNA (pDNA) can be made using a commercially available material, such as PEI-MAX (Polysciences, Inc., Warrington, Pa.). To make a carrier material comprised of PEI-MAX nanoparticles, for example, a solution of about 10 mM PEI MAX can be prepared in nuclease-free water at a pH of about 7.2. The solution can then be sterilized using a filter. Next, PEI-MAX/pDNA nanoparticles can be made by determining a desired N/P ratio according to the following equation:

(X μg DNA)×(3 nmol P/μg DNA)×(Y nmol N/nmol P)×(1 μl/10 nmol N)

where X is the amount of DNA desired and Y is the desired N/P ratio. Using a 24-well plate, about 1 μg of DNA and about 6 μl of PEI-MAX per well can be used, i.e., an N/P ratio of about 20. For example, about 1 μg of pDNA can be mixed with serum-free media for a final volume of about 50 μl, and about 6 μl of PEI-MAX nanoparticles can then be mixed with about 44 μl of serum-free media. The mixture can then sit at about room temperature for about 10 minutes. The PEI-MAX can be added to the pDNA all at once and then vortexed for about 10 seconds. The resultant PEI-MAX/pDNA nanoparticles can then sit at room temperature for about 30 minutes before further use.

In yet another example, a carrier material including pDNA can be made using a commercially available material, such as JETPEI (Polyplus-Transfection SA, Illkirch, France) according to the manufacturer's protocol. Briefly, for example, JETPEI/pDNA nanoparticles can be made by first determining a desired N/P ratio according to the equation provided above. After determining the desired N/P ratio, a 24-well plate can be selected. For each well, about 0.5 to about 1 μg of pDNA can be diluted into about 50 μl of about 150 mM NaCl. The 24-well plate can then be vortexed gently and spun down briefly. Next, about 1 μl to about 2 μl of JETPEI solution can be added into about 50 μl of about 150 mM NaCl (for each well), vortexed gently, and then spun down briefly. About 50 μl of the JETPEI solution can then be added to the pDNA solution all at once. The resultant solution can be vortexed immediately and then spun down briefly to bring drops to the bottom of the wells. The plate can then be incubated for between about 15 and 30 minutes at about room temperature.

In another example, a carrier material capable of including an interfering RNA molecule can be prepared using a commercially available material, such as LIPOFECTAMINE 2000 (Invitrogen Corp., Carlsbad, Calif.) according to the manufacturer's protocol. Briefly, for example, about 20 μmol of interfering RNA molecules can be diluted in about 50 μl of OPTIMEM I Reduced Serum Medium (Invitrogen Corp., Carlsbad, Calif.) without serum (for a final concentration of the interfering RNA molecules of about 33 nM when mixed with progenitor cells). After mixing the solution gently, about 1 μl of LIPOFECTAMINE 2000 can be diluted in about 50 μl of OPTIMEM I Reduced Serum Medium. The resultant solution can then be mixed gently and incubated for about 5 minutes at about room temperature. Within about 25 minutes after the 5 minute incubation, the diluted interfering RNA molecules can be combined with the diluted LIPOFECTAMINE 2000, mixed gently, and then incubated for about 20 minutes at about room temperature.

In another example, a carrier material capable of including an interfering RNA molecule can be prepared using a commercially available material, such as INTERFERIN (Polyplus-Transfection SA, Illkirch, France) according to the manufacturer's protocol. Briefly, for example, a 24-well plate can be selected. For each well, about 0.6 pmol of interfering RNA molecules can be diluted into about 100 μl of medium without serum or in OPTIMEM. The wells can then be vortexed gently. Next, about 2 μl of INTERFERIN can be added to the solution containing the interfering RNA molecules and then homogenized immediately for about 10 seconds. The 24-well plate can be incubated for about 10 minutes at about room temperature to allow complexes to form between the interfering RNA molecules and the INTERFERIN. This step should not exceed about 30 minutes. During complex formation, growth medium can be removed and about 0.5 ml of fresh pre-warmed completed media added per well.

After preparing the at least one carrier material, the carrier material can be mixed with the polymeric macro- or micro-scaffold at 16. The carrier material can be mixed with the polymeric macro- or micro-scaffold at a desired concentration and for an appropriate amount of time using, for example, mechanical or tactile force. For example, calcium phosphate nanoparticles coated with DNA can be prepared (described above) and then mixed with about a 2% alginate solution (described above) at a concentration of about 10% v/v. The alginate can then be cross-linked with a slurry of calcium sulfate and cast into a container to form a mold having a desired shape. After about 20 minutes, the molded composition can be removed from the container and formed into the bioresorbable implant composition or stored for later use.

At step 18, at least one cell can then be prepared. Any known method may be employed to harvest, maintain, expand, and prepare cells for use in the present invention. For example, MSCs, which can differentiate into a variety of mesenchymal or connective tissues (including, for example, adipose, osseous, cartilagenous, elastic, and fibrous connective tissues), can be isolated, purified, and replicated according to known techniques (see, e.g., Caplan et al., U.S. Pat. No. 5,486,359 and Caplan et al., U.S. Pat. No. 5,226,914, each of which is incorporated herein by reference). Cells can be expanded ex vivo prior to introduction into the polymeric macro- or micro-scaffold. For example, CD34⁺ MSCs can be derived from the bone marrow of a subject and then cultured in FGF-2, as described by Solchaga et al., J Cell Physiol., 203(2):398-409 (2005). Culturing the MSCs in FGF-2 can increase the proliferative potential of the MSCs. It will be appreciated that the at least one cell can be seeded or mixed into the polymeric macro- or micro-scaffold before or after the bioactive agent and/or carrier material has been mixed with the polymeric macro- or micro-scaffold.

After preparing the cells, the bioresorbable implant composition can be formed at 20. To form the bioresorbable implant composition, cells may be dispersed uniformly within the polymeric macro- or micro-scaffold or, alternatively, dispersed such that different densities and/or spatial distributions of different or the same cells are dispersed within different portions of the polymeric macro- or micro-scaffold. It will be appreciated, however, that the cells may be seeded before incorporation of the carrier material or, alternatively, after or at the same time as incorporation of the carrier material.

Generally, cells can be introduced into the polymeric macro- or micro-scaffold in vitro, although in vivo seeding approaches can optionally or additionally be employed. Cells may be mixed with the polymeric macro- or micro-scaffold and cultured in an adequate growth (or storage) medium to ensure cell viability. If the composition is to be implanted for use in vivo after in vitro seeding, for example, sufficient growth medium may be supplied to ensure viability throughout the composition during in vitro culture prior to in vivo application. Once the composition has been implanted, the nutritional requirements of the cells can be met by the circulating fluids of the host subject.

Any available method may be employed to introduce the cells into the polymeric macro- or micro-scaffold. For example, cells may be injected into the polymeric macro- or micro-scaffold (preferably in combination with growth medium) or may be introduced by other means, such as pressure, vacuum, osmosis, or manual mixing. Alternatively or additionally, cells may be layered on the polymeric macro- or micro-scaffold, or the polymeric macro- or micro-scaffold may be dipped into a cell suspension and allowed to remain there under conditions and for a time sufficient for the cells to incorporate within or attach to the macro- or micro-scaffold. Generally, it is desirable to avoid excessive manual manipulation of the cells in order to minimize cell death during the impregnation procedure. For example, in some situations it may not be desirable to manually mix or knead the cells with the polymeric macro- or micro-scaffold; however, such an approach may be useful in those cases in which a sufficient number of cells will survive the procedure. Cells can also be introduced into the polymeric macro- or micro-scaffold in vivo simply by placing the macro- or micro-scaffold in the subject adjacent a source of desired cells. Bioactive agents released from the macro- or micro-scaffold may also recruit local cells, cells in the circulation, or cells at a distance from the implantation or injection site.

As those of ordinary skill in the art will appreciate, the number of cells to be introduced into the polymeric macro- or micro-scaffold will vary based on the intended application of the polymeric macro- or micro-scaffold and on the type of cell used. Where dividing autologous cells are being introduced by injection or mixing into the polymeric macro- or micro-scaffold, for example, a lower number of cells can be used. Alternatively, where non-dividing cells are being introduced by injection or mixing into the polymeric macro- or micro-scaffold, a larger number of cells may be required.

In an example of the present invention, CD34⁺ MSCs may be derived from the bone marrow of a subject and then cultured in FGF-2 (as described above). After culture of the MSCs with FGF-2, the MSCs may be mixed with the polymeric macro- or micro-scaffold, such as a peptide-modified alginate macro- or micro-scaffold (described above) for a time sufficient to permit incorporation of the MSCs onto or within the macro- or micro-scaffold. The MSC- macro- or micro-scaffold composition can then be mixed with first and second carrier materials that respectively include first and second bioactive agents. It will be appreciated, however, that the polymeric macro- or micro-scaffold may be exposed to the first and second carrier materials prior to combination with the MSCs.

It will be appreciated that a nanoparticle or microparticle comprising the carrier material and at least one bioactive agent can be incorporated into the cells (e.g., by transfection) prior to seeding the progenitor cells into or onto the polymeric macro- or micro-scaffold. Where PEI-MAX/pDNA nanoparticles have been formed (as described above), 24-well plates can be seeded about 1 day prior to transfection at a desired density (e.g., about 1.5×10⁵ cells/well) and then incubated overnight in complete medium without antibiotics. The serum-containing media can be removed from the cells and replaced with about 400 μl of serum-free media. About 100 μl of the PEI-MAX/pDNA nanoparticles can then be added to each well, followed by incubation for about 4 to 6 hours at about room temperature. The serum-free media can then be removed and replaced with serum-containing media. The progenitor cells can then be assayed to verify effective transfection of the pDNA about 24 to about 72 hours following transfection.

Where JETPEI/pDNA nanoparticles have been formed (as described above), 24-well plates can be seeded with progenitor cells about 1 day prior to transfection at a desired density (e.g., about 1×10⁵ cells/well to about 2×10⁵ cells/well) and then incubated overnight in serum-containing media. About 100 μl of the JETPEI/pDNA nanoparticles can then be added drop-wise onto the serum-containing media in each well and homogenized by gently swirling the plate. The plate can then be incubated at about 37° C. and about 5% CO₂ in a humidified atmosphere for about 24 to about 48 hours. The progenitor cells can be assayed to verify effective transfection of the pDNA, collected by centrifugation at about 400 g, and then resuspended in a desired medium or buffer.

Where LIPOFECTAMINE 2000/interfering RNA molecule complexes have been formed (as described above), the complexes can be added to each well of a 24-well plate containing progenitor cells and medium. The 24-well plate can then be mixed gently by rocking the plate back and forth. Next, the progenitor cells can be incubated at about 37° C. in a CO₂ incubator for about 24 to about 96 hours or until an appropriate assay is performed to verify effective transfection of the interfering RNA molecules. The medium can be changed after about 4 to about 6 hours.

Where INTERFERIN/interfering RNA molecule complexes have been formed (as described above), about 100 μl of a solution comprising the complexes can be added to the wells of a 24-well plate and then homogenized by gently swirling the plate. The final volume per well can be about 600 μl with a concentration of the interfering RNA molecules of about 1 nM. The plate can be incubated at about 37° C. in an appropriate CO₂ atmosphere. Target gene silencing can be assayed between about 24 and about 72 hours for mRNA levels, and about 24 to about 96 hours for polypeptides or proteins.

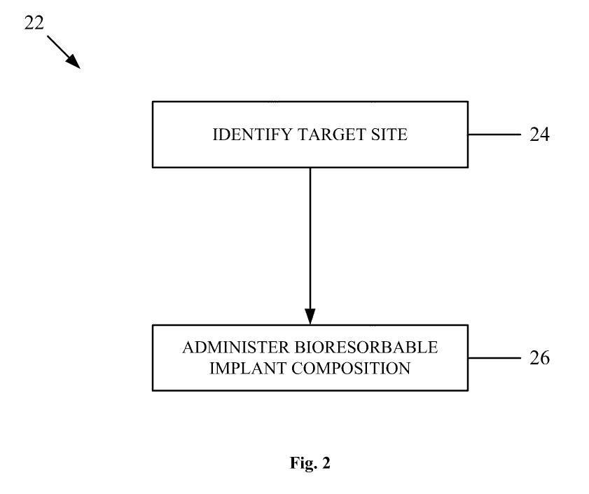

FIG. 2 is a flow diagram illustrating a method 22 for promoting tissue growth in a subject in accordance with another aspect of the invention. In the method 22, at step 24 a target site is identified. The target site can comprise a tissue defect (e.g., cartilage and/or bone defect) in which promotion of new tissue (e.g., cartilage and/or bone) is desired. The target site can also comprise a diseased location (e.g., tumor). Methods for identifying tissue defects and disease locations are known in the art and can include, for example, various imaging modalities, such as CT, MRI, and X-ray.

The tissue defect can include a defect caused by the destruction of bone or cartilage. For example, one type of cartilage defect can include a joint surface defect. Joint surface defects can be the result of a physical injury to one or more joints or, alternatively, a result of genetic or environmental factors. Most frequently, but not exclusively, such a defect will occur in the knee and will be caused by trauma, ligamentous instability, malalignment of the extremity, meniscectomy, failed aci or mosaicplasty procedures, primary osteochondritis dessecans, osteoarthritis (early osteoarthritis or unicompartimental osteochondral defects), or tissue removal (e.g., due to cancer). Examples of bone defects can include any structural and/or functional skeletal abnormalities. Non-limiting examples of bone defects can include those associated with vertebral body or disc injury/destruction, spinal fusion, injured meniscus, avascular necrosis, cranio-facial repair/reconstruction (including dental repair/reconstruction), osteoarthritis, osteosclerosis, osteoporosis, implant fixation, trauma, and other inheritable or acquired bone disorders and diseases.

Tissue defects can also include cartilage defects. Where a tissue defect comprises a cartilage defect, the cartilage defect may also be referred to as an osteochondral defect when there is damage to articular cartilage and underlying (subchondral) bone. Usually, osteochondral defects appear on specific weight-bearing spots at the ends of the thighbone, shinbone, and the back of the kneecap. Cartilage defects in the context of the present invention should also be understood to comprise those conditions where surgical repair of cartilage is required, such as cosmetic surgery (e.g., nose, ear). Thus, cartilage defects can occur anywhere in the body where cartilage formation is disrupted, where cartilage is damaged or non-existent due to a genetic defect, where cartilage is important for the structure or functioning of an organ (e.g., structures such as menisci, the ear, the nose, the larynx, the trachea, the bronchi, structures of the heart valves, part of the costae, synchondroses, enthuses, etc.), and/or where cartilage is removed due to cancer, for example.

After identifying a target site, such as a cranio-facial cartilage defect of the nose at step 26, a bioresorbable implant composition can be administered to the target site. The bioresorbable implant composition can first be prepared according to the method 10 described above. In one example of the present invention, a bioresorbable implant composition comprising a polypeptide-modified alginate macro- or micro-scaffold, at least one CD34⁺ MSC, and first and second carrier materials respectively including first and second bioactive agents incorporated dispersed on or within the alginate macro- or micro-scaffold may be prepared (as described above). The alginate macro- or micro-scaffold can be modified with at least one polypeptide having the amino acid sequence of SEQ ID NO: 1 to facilitate incorporation of at least one CD34⁺ MSC into or onto the macro- or micro-scaffold. Each of the first and second carrier materials can be comprised of PLGA and may be prepared as described above. For example, the first carrier material may comprise a greater mixture of L and D stereoisomers to increase the degradation rate of the first carrier material. Additionally, the second carrier material may comprise a lower mixture of L and D stereoisomers (as compared to the first carrier material) so that the second carrier material has a slower degradation rate when exposed to physiological conditions.

The first and second bioactive agents may then be impregnated into and/or coated onto the first and second carrier materials, respectively. The first bioactive agent can comprise a growth factor (e.g., TGF-β, VEGF, and/or FGF-2) or, alternatively, a plasmid including a polynucleotide that encodes a growth factor (e.g., TGF-β, VEGF, and/or FGF-2). Similarly, the second bioactive agent can comprise a growth factor (e.g., IGF-I and/or BMP-2) or, alternatively, a plasmid including a polynucleotide that encodes a growth factor (e.g., IGF-I and/or BMP-2). The carrier materials may be mixed with the alginate macro- or micro-scaffold in an amount and for a time sufficient to permit incorporation of the carrier materials into and/or onto the macro- or micro-scaffold. It should be appreciated that in order to regenerate bone, FGF-2 and/or VEGF can be incorporated into the first bioactive agent and BMP-2 (or a DNA plasmid encoding BMP-2) can be incorporated into first and second carrier materials (e.g., made of PLGA).

Next, at least one CD34⁺ MSC may be obtained from the bone marrow of the subject and then expanded ex vivo using a growth factor, such as FGF-2. After expanding the cells to a desired concentration, the cells can be seeded into and/or onto the alginate macro- or micro-scaffold trix (as described above) to form the bioresorbable implant composition. The bioresorbable implant composition may then be loaded into a syringe or other similar device and injected or implanted into the tissue defect of the subject. Upon injection or implantation into the subject, the bioresorbable implant composition may be formed into the shape of the tissue defect using, for example, tactile means. Alternatively, the implant may be formed into a specific shape prior to injection or implantation into the subject.

After implanting the bioresorbable implant composition in the subject, the first carrier material may begin to degrade faster than the second carrier material (or allow for increased diffusion relative to the first carrier material) and thereby release the growth factor (e.g., TGF-β) or the polynucleotide encoding the growth factor. Release of TGF-β from the first carrier material can promote early CD34⁺ MSC commitment to a particular lineage (e.g., chondrogenic lineage). As the cells proliferate, the second carrier material may degrade more slowly than the first carrier material and thereby release the other growth factor (e.g., IGF-I) or the polynucleotide encoding the other growth factor at a slower rate. Release of IGF-I from the second carrier material can promote differentiation of the cells into more mature cells (e.g., chondroprogenitor cells). The continued release of IGF-I, along with other growth and/or differentiation factors expressed by the cells (i.e., the cells comprising the bioresorbable implant composition as well as the cells surrounding the tissue defect), can promote development of mature cells (e.g., chondrocytes) capable of generating new tissue (e.g., cartilage) for repair of the tissue defect.

In another example of the method 22, a bioresorbable implant composition comprising a polymeric macro- or micro-scaffold, at least one progenitor cell, and first and second calcium phosphate nanoparticle including first and second bioactive agents, respectively, can be administered to a target site step 26. The polymeric macro- or micro-scaffold can comprise a polypeptide-modified alginate macro- or micro-scaffold, as described above. The first bioactive agent can comprise plasmid DNA (e.g., a DNA plasmid encoding VEGF and/or FGF-2), and the second bioactive agent can comprise plasmid DNA (e.g., a DNA plasmid encoding BMP-2). The first and second bioactive agents can be at least partially coated onto the surface of first and second calcium phosphate nanoparticles (as described above).

After forming the first and second calcium phosphate nanoparticles, the nanoparticles may be mixed with the alginate macro- or micro-scaffold as described above. At least one progenitor cell, such as a CD34⁺ MSC may be obtained from the subject. As described above, the CD34⁺ cells can then expanded ex vivo using a growth factor, such as FGF-2. After expanding the CD34⁺ MSCs to a desired concentration, the cells can be seeded into and/or onto the polymeric macro- or micro-scaffold to form the bioresorbable implant composition. The bioresorbable implant composition can then be loaded into a syringe (or other similar device), the alginate cross-linked, and then injected or implanted into the tissue defect. Upon injection or implantation, the bioresorbable implant composition may be formed into the shape of the tissue defect using, for example, tactile means.

After implanting the bioresorbable implant composition in the subject, the first calcium phosphate nanoparticle may begin to degrade faster than the second calcium phosphate nanoparticle (or allow for increased diffusion as compared to the first calcium phosphate nanoparticle) and thereby release the first bioactive agent (e.g., the DNA plasmid encoding FGF-2). The CD34⁺ MSCs can then uptake the released first bioactive agent and begin to express FGF-2, in turn promoting cell proliferation and angiogenesis. As the cells proliferate, the second calcium phosphate nanoparticle may begin to disintegrate or diffuse and thereby release the second bioactive agent (e.g., the DNA plasmid encoding BMP-2). The cells may then uptake the released second bioactive agent and begin to express BMP-2. Expression of BMP-2 may then promote differentiation of the cells into more mature cells (e.g., osteoblasts). The continued release of the bioactive agents, along with other growth and/or differentiation factors expressed by the cells, can promote development of mature cells (e.g., osteoblasts) capable of generating new tissue (e.g., bone) for repair of the tissue defect.

In yet another example of the method 22, a bioresorbable implant composition comprising a polymeric macro- or micro-scaffold, at least one progenitor cell, and at least one carrier material including at least one interfering RNA molecule can be administered to a target site at 26. The polymeric macro- or micro-scaffold can comprise a polypeptide-modified alginate macro- or micro-scaffold, as described above. The at least one carrier material can comprise a calcium phosphate nanoparticle or, alternatively, a microparticle or nanoparticle formed from a synthetic polymer, such as polyethylenimine (PEI). The interfering RNA molecule can comprise an siRNA molecule capable of inhibiting or reducing expression of a target mRNA, such as an mRNA encoding GNAS. The interfering RNA molecule can be at least partially coated onto the surface of the at least one carrier material (as described above). After forming the carrier material, the carrier material can be mixed with the alginate macro- or micro-scaffold as described above. At least one progenitor cell, such as a CD34⁺ MSC may then be obtained from the subject. As described above, the CD34⁺ cells can be expanded ex vivo using a growth factor, such as FGF-2. After expanding the CD34⁺ MSCs to a desired concentration, the cells can be seeded into and/or onto the polymeric macro- or micro-scaffold to form the bioresorbable implant composition. The bioresorbable implant composition can then be loaded into a syringe or other similar device and injected or implanted into the tissue defect. Upon injection or implantation, the bioresorbable implant composition may be formed into the shape of the tissue defect using, for example, tactile means.

After implanting the bioresorbable implant composition in the subject, a function and/or characteristic of at least one of the CD34⁺ MSCs can be modulated through the process of RNA interference (RNAi). Briefly, RNAi is a process by which double-stranded RNA (dsRNA) can be used to prevent or reduce gene expression. While not wanting to be bound by theory, RNAi can begin with the cleavage of longer dsRNAs into siRNAs by an RNaseIII-like enzyme (i.e., dicer). siRNAs are dsRNAs that are usually about 19 to 28 polynucleotides, or about 20 to 25 polynucleotides, or about 21 to 22 polynucleotides in length, and contain 2-nucleotide 3′ overhangs and 5′ phosphate and 3′ hydroxyl termini.

Once the bioresorbable implant composition has been implanted, carrier molecules can be endocytosed by the CD34⁺ MSCs. Once inside of the cells, the endosomes encapsulating the carrier molecules may be disrupted and the carrier molecules thereby released into the cytosol of the cell. The released carrier molecules can then aid in the functional activity of the siRNA molecules of mRNA degradation or begin to degrade and release the siRNA molecules coated thereon. As described above, the process of RNAi may then take place inside of the cells. In the context of the present example, the activity of the GNAS or GNAS 1 gene can be modulated (i.e., down-regulated) by the released siRNA molecules by way of the Cbfa1 pathway. For example, the activity of Cbfa1 is regulated by the α chain of heterotrimeric G protein, Gsα, and is transcribed by the gene GNAS 1. The heterotrimeric G protein (Gs) couples heptahelical receptors for hormones, such as parathyroid hormone to stimulate adenylyl cyclase. Reduction in Gsα induces osteogenic differentiation in human MSCs. The activation of adenylyl cyclase or the adenosine monophosphate (cAMP)-dependent signaling pathway inhibits osteogenic differentiation by means of proteasomal degradation of Cbfa1 via the ubiquitin-proteasome pathway. Down-regulation of the GNAS or GNAS 1 gene via RNAi can lead to the increased expression of Cbfa1. Increased expression of Cbfa1 can then lead to increased production of bone-differentiating genes and, in turn, result in regulated osteogenic differentiation of MSCs.

In addition to siRNA molecules, other interfering RNA molecules and RNA-like molecules can interact with the RISC and silence gene expression. Non-limiting examples of other interfering RNA molecules that can interact with the RISC can include short hairpin RNAs (shRNAs), shRNA molecules containing one or more chemically modified nucleotides, single-stranded siRNAs, microRNAs, dicer-substrate 27-mer duplexes, one or more non-nucleotides, one or more deoxyribonucleotides, DNA encoding for any of these RNA or RNA-like molecules and/or one or more non-phosphodiester linkages.

One having ordinary skill in the art will appreciate various changes and modifications to the present invention. For example, the components of the bioresorbable implant composition may be combined in any desired manner, and not necessarily in the order described above. Further, it will be appreciated that the present invention may also be used to mobilize endogenous cells surrounding a tissue defect to guide their infiltration into a particular defect and subsequent function and/or differentiation.

The following examples are for the purpose of illustration only and are not intended to limit the scope of the claims, which are appended hereto.

### EXAMPLE 1

### Alginate Preparation

Sodium alginate powder (FMC Biopolymers, Princeton, N.J.) was lyophilized until dry, purified by dialysis for 4 days, subjected to activated charcoal treatment, and then sterilized through a 0.22 μm filter. Some of the alginate was subjected to gamma irradiation at 5 MRad (Phoenix Lab, University of Michigan, Ann Arbor). The molecular weight was found to be 37,000 g/mol for irradiated alginate, and 121,000 g/mol for non-irradiated alginate as determined by SEC-MALS (FMC Biopolymers). A polypeptide having the amino acid sequence of SEQ ID NO: 1 (Commonwealth Biotechnologies, Richmond, Va.) was covalently coupled to the irradiated alginate as described by Luo D. et al., Nat. Biotechnol., 18(1):33-37 (2000). The plasmid pcDNA3.1/Hygro/lacZ was obtained from Invitrogen (Carlsbad, Calif.). MC3T3-E1 Subclone 4 (ATCC #CRL-2593) cells were obtained from American Type Culture Collection (Manassas, Va.). Phosphate buffered saline (PBS) and (α-MEM were obtained from Hyclone (Logan, Utah). All other chemicals were obtained from Fisher Scientific (Fairlawn, N.J.).

### EXAMPLE 2

### Preparation and Characterization of Calcium-Phosphate DNA Nanoparticles

Two types of Calcium-Phosphate DNA Nanoparticles were fabricated: calcium phosphate core with DNA coating, and calcium phosphate-DNA core with BSA coating. CaP core-DNA coated particles were created by a slight modification to the previously described method of Sokolova, V. V. et al., Biomaterials, 27(16):3147-3153 (2006). Equal volumes of 18.7 mM CaCl₂ (pH 9) and 11.23 mM Na₂HPO₄ (pH 9) were added simultaneously to a tube with a magnetic stir bar. The solution was mixed for 30 seconds and 200 μg of DNA was added to quench the crystallization by coating the crystals. CaP-DNA core/BSA coated particles were created by a modification of the method described by Li, Y et al., Int J Pharm., 269(1):61-70 (2004). 120 μg DNA was mixed with 100 μl 2 M CaCl₂. This solution was added drop-wise to 1 ml of 2×HBS (pH 7) while stirring. Then, 780 μl of distilled water was immediately added. The mixture was stirred at room temperature for 30 minutes, and then 200 μg of bovine serum albumin was added to halt the crystallization.

The DNA encapsulation efficiency of each particle type was determined by centrifugation of the particles at 17,000 rpm for 30 minutes, followed by measurement of the DNA in the supernatant by PicoGreen. Plasmid DNA at known concentrations was used to construct the standard curve.

The size of the particles was determined by transmission electron microscopy (TEM). Particles were freshly prepared (n=3 preparations for each type), and a sample of each was diluted 1:50 with distilled water, spotted onto a nickel formvar grid, and allowed to dry at 37° C. The remaining particles were kept for two weeks, and samples were taken at one and two weeks for imaging. From these images, particle diameters were calculated using ImageJ software.

### EXAMPLE 3

### In vitro Cell Transfection Using Calcium-Phosphate DNA Nanoparticles

CaP core-DNA coated and CaP-DNA core-BSA coated NPs were freshly prepared using DNA encoding for lacZ. MC3T3-E1 cells were seeded the day before transfection. For transfection, the cells were rinsed once with PBS, followed by the addition of serum-free media containing 10% v/v of NPs. The cells were incubated for 5 hours at 37° C., and then the media was removed and replaced with complete medium (α-MEM+10% FBS). 48 hours post-transfection, the cells were rinsed with PBS, fixed with 0.2% glutaraldehyde, and stained with X-Gal to assay lacZ expression. 24 hours after staining, the cells were rinsed with PBS and examined under the microscope to determine transfection efficiency.

### EXAMPLE 4

### In vitro DNA Release from Alginate Hydrogels

CaP particles were freshly prepared, and mixed with a 2% alginate solution at a concentration of 10% v/v. The alginate was then crosslinked with a slurry of calcium sulfate and cast between two glass plates spaced 0.75 mm apart. After 20 minutes, 10 mm diameter disks were cut out and transferred to PBS containing calcium and magnesium. The disks were incubated at 37° C. under gentle agitation. Release samples were taken periodically by removing the PBS and replacing with fresh PBS. The released DNA was measured using PicoGreen.

### EXAMPLE 5

### In vivo Study

CaP particles were freshly prepared and mixed into a 2% GRGDSP-alginate solution, followed by the addition of MC3T3 cells at a final concentration of 24E6 cells/ml. The alginate was crosslinked as described above with calcium sulfate, and kept in a syringe on ice until injection. 200 μl of each experimental condition (N=2) was injected subcutaneously though an 18-gauge needle into the backs of anesthetized 5-week-old male C.B-17 SCID mice (Harlan, Indianapolis, Ind.). Implants were harvested, fixed, and processed histologically at 2.5 and 6 weeks post-injection. Slides were stained with hematoxylin and eosin (H&E) or Goldner's Trichrome.

### EXAMPLE 6

### Calcium Phosphate-DNA Nanoparticles Characterization and in vitro Transfection Capacity

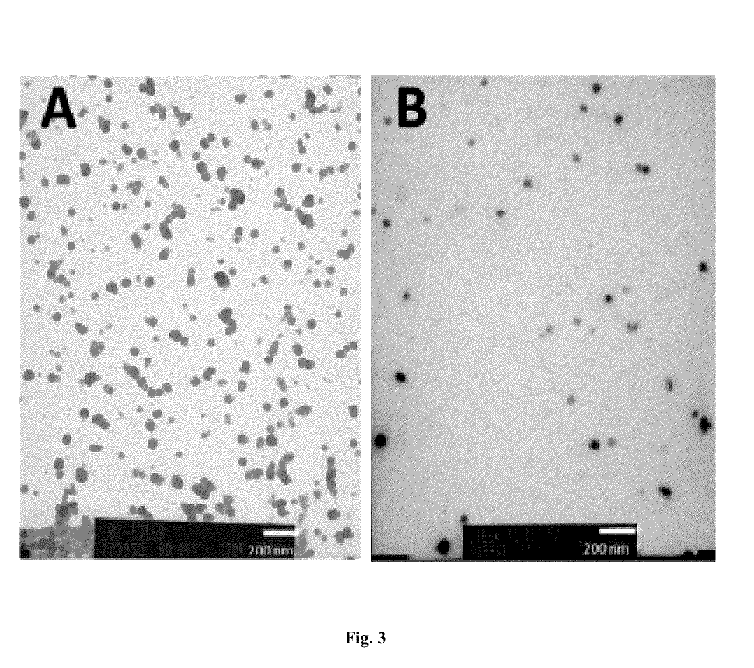

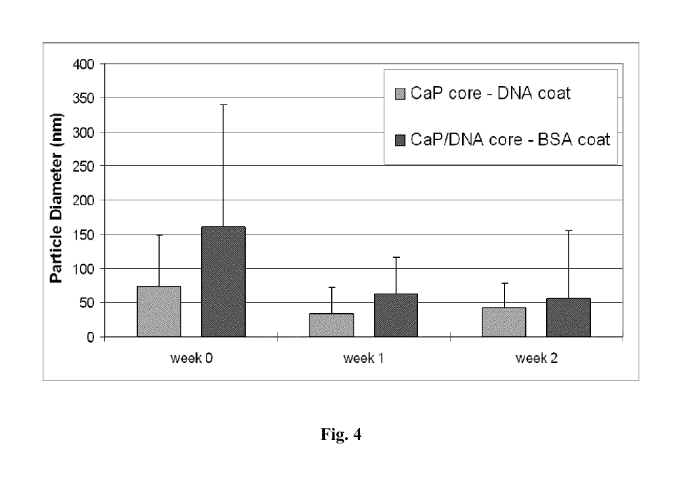

The size of the CaP-DNA nanoparticles was quantified to determine their stability over time. The size and morphology of both types of NPs were examined at 0, 1, and 2 weeks post-fabrication by transmission electron microscopy. Representative images are depicted in FIGS. 3A-B. The particle sizes were determined, and as shown in Table 1 and FIG. 4, the particles did not grow or aggregate over time. At time zero, the CaP core-DNA coat NPs were 75 nm in diameter on average, while the CaP-DNA core-BSA coat NPs were found to be 161 nm on average. Additionally, the DNA incorporation efficiency was determined to be 66.5%+/−3.5% for CaP core-DNA coat NPs, and 79.5%+/−16.2% for CaP-DNA core-BSA coat NPs.

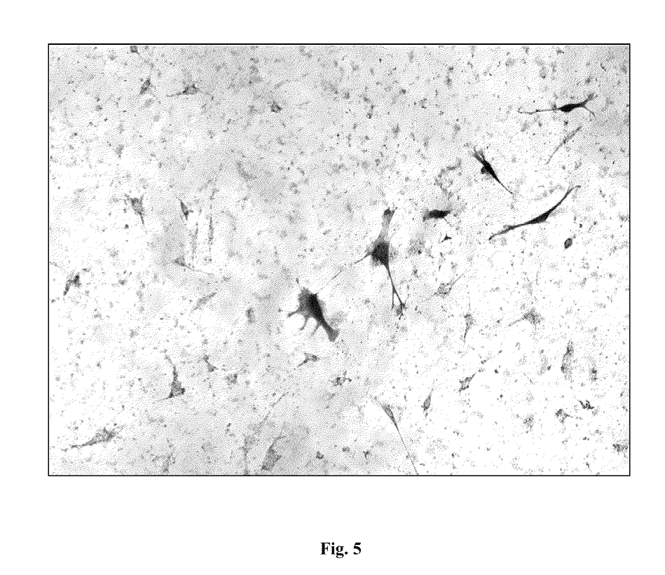

The ability of these particles to transfect preosteoblast cells in vitro was examined using DNA encoding for lacZ (FIG. 5). The transfection efficiency was low; around 1% of cells stained positively for lacZ expression. However, we were able to verify that both particle types had the ability to transfect preosteoblast cells.

TABLE 1

| | CaP core-DNA coat | CaP-DNA core-BSA coat |
|---|---|---|
| Week 0 | 74.77 +/− 73.1 | 160.75 +/− 178.8 |
| Week 1 | 34.25 +/− 38.5 | 62.86 +/− 53.5 |
| Week 2 | 41.55 +/− 37.3 | 56.40 +/− 98.7 |

### EXAMPLE 7

### Release of CaP-DNA NPs From Alginate Hydrogels

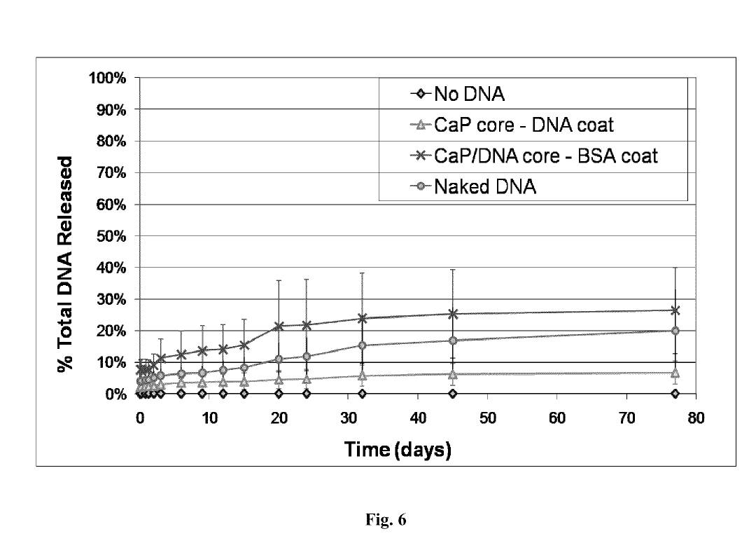

The in vitro release of naked DNA and both types of CaP-DNA NPs from alginate hydrogels over the course of 2 weeks was quantified. As seen in FIG. 6, all conditions show sustained release of pDNA up to two months. pDNA from alginate containing CaP-DNA core-BSA coat particles was released most rapidly, followed by naked DNA, then by CaP core-DNA coat particles. A large fraction of DNA remained in the hydrogels originally containing DNA, which would be available to transfect cells incorporated in the hydrogels with the NPs.

### EXAMPLE 8

### In vivo Study

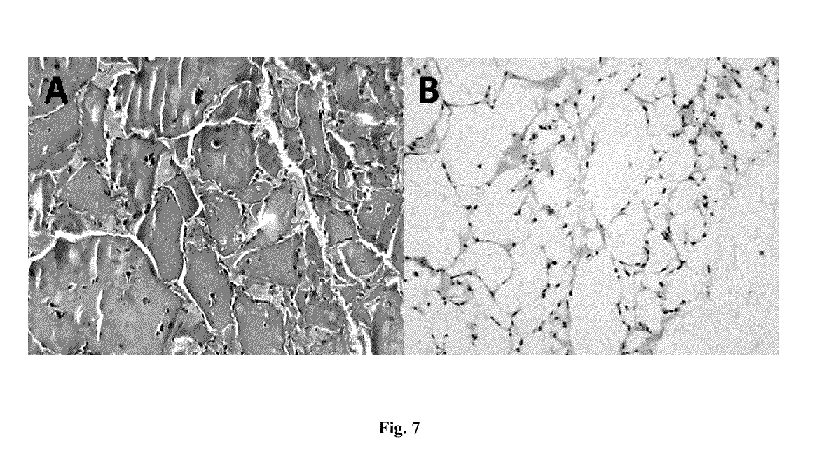

For the in vivo study, we used low MW alginate with RGD modification. As shown previously, irradiated alginate degrades more rapidly in vivo, and the RGD amino acid sequence allows cells incorporated within the alginate to adhere to the hydrogel (Alsberg, E. et al., J Dent Res., 82(11):903-908, 2003). CaP-DNA NPs and preosteoblast cells were mixed into irradiated alginate modified with the cellular adhesive polypeptide (SEQ ID NO: 1). Additionally, as a control, cells in the modified alginate without particles or DNA were examined to obtain a background level of bone formation due to the cells alone, if any. Implants were harvested at 2.5 and 6 weeks to examine whether any bone formation had occurred. The implants were processed for histology and stained with either H&E or Goldner's Trichrome. Bone tissue appears a pink color using H&E, and a light green color using Goldner's Trichrome. The alginate stains purple with H&E, and does not stain with Goldner's Trichrome. In the samples with cells only (i.e., no NPs or DNA), no bony tissue was seen at any time point. This was also the case for samples with CaP-DNA core-BSA coat particles. However, for the CaP core-DNA coat particles, bony tissue as shown in FIG. 7 was found in half of the implants.

### EXAMPLE 9

### Synthesis of PLGA Microspheres Including BSA

PLGA microspheres including BSA were synthesized by preparing the following solutions: (a) 5% w/v PLGA in ethyl acetate; (b) an aqueous solution including BSA; (c) a secondary emulsion solution comprising 5% w/v PVA (MW 9-10 KDa), and 7% ethyl acetate in water; and (d) an extraction solution comprising 0.3% w/v PVA and 7% ethyl acetate in water. To prepare a primary emulsion, 100 μl of the BSA solution was added to about 1 ml of the PLGA solution and then sonicated for 15 seconds at a frequency of 20 W. This was done in an ice bath to avoid overheating. Next, 1 ml of the PVA solution was added to the primary emulsion and then vortexed at a maximum speed for 15 seconds. The secondary emulsion was then poured into 200 ml of the extraction solution and stirred continuously for 3 hours. The extraction solution with the microspheres was filtered through a vacuum. The microspheres were then rinsed off of the filter with water, poured into a 50 ml conical tube through a 70 μm cell strainer, and centrifuged for 10 minutes at 7,000 rpm. The supernatant was discarded and the microspheres resuspended in water. This step was repeated twice. The collected microspheres were then flash frozen in liquid nitrogen for 5 minutes, followed by lyophilization.

### EXAMPLE 10

### Synthesis of PLGA Microspheres Including BSA

PLGA microspheres including BSA were synthesized by preparing the following solutions: (a) 5% w/v PLGA in ethyl acetate; (b) an aqueous solution including BSA; and (c) 0.1% to 3% PVA w/v (MW 30-70 KDa) for the secondary emulsion. To prepare a primary emulsion, 1 ml of the aqueous BSA solution was added to 10 ml of the PLGA solution and then sonicated for 15 seconds at a frequency of 20 W. This was done in an ice bath to avoid overheating. Next, the primary emulsion was poured into 200 ml of the PVA solution and then homogenized at 10,000 rpm for 1 minute using an ice bath to avoid overheating. The secondary emulsion was stirred continuously for 3 hours, followed by filtering of the extraction solution (with the microspheres) through a vacuum filter. The microspheres were rinsed off of the filter with water, poured into a 50 ml conical tube through a 70 μm cell strainer, and centrifuged for 10 minutes at 7,000 rpm. The supernatant was discarded and the microspheres resuspended in water. This step was repeated twice. The collected microspheres were then flash frozen in liquid nitrogen for 5 minutes, followed by lyophilization.

From the above description of the invention, those skilled in the art will perceive improvements, changes and modifications. For example, it will be appreciated that the order of steps shown in FIGS. 2 and 3 are illustrative only and are not intended to limit the method to the order of steps described herein. Such improvements, changes, and modifications are within the skill of the art and are intended to be covered by the appended claims. All patent publications and references cited in the present application are herein incorporated by reference in their entirety.

Having described the invention, the following is claimed:

1. A bioresorbable implant composition comprising:
a polymeric macro- or micro-scaffold; and
first and second bioactive agents incorporated on or within the polymeric macro- or micro-scaffold, the first and second bioactive agents modulating a different function and/or characteristic of a cell.

2. The bioresorbable implant composition of claim 1 further comprising at least one cell dispersed within or on the polymeric macro- or micro-scaffold.

3. The bioresorbable implant composition of claim 2 further comprising first and second carrier materials dispersed on or within the polymeric macro- or micro-scaffold, the first and second carrier materials respectively including first and second bioactive agents, the first and second carrier materials differentially releasing the first and second bioactive agents to modulate a function and/or characteristic of the at least one cell.

4. The bioresorbable implant composition of claim 3, the first carrier material releasing the first bioactive agent with a different release profile than the release profile of the second bioactive agent from the second carrier material.

5. The bioresorbable implant composition of claim 3, the first and second carrier materials comprising a degradable carrier material selected from the group consisting of calcium phosphate nanoparticles, PLGA microparticles, and combinations thereof.

6. The bioresorbable implant composition of claim 3, the first carrier material degrading or diffusing before degradation or diffusion of the second carrier material.

7. The bioresorbable implant composition of claim 3, the first carrier material allowing for more rapid release or diffusion of the first bioactive agent as compared to the second carrier material.

8. The bioresorbable implant composition of claim 2, the polymeric macro- or micro-scaffold further including at least one attachment molecule for facilitating attachment of the at least one cell to the polymeric macro- or micro-scaffold.

9. The bioresorbable implant composition of claim 2, the at least one cell being exposed in vitro or in situ to at least one growth factor to increase the proliferative potential and/or differentiation of the at least one cell.

10. The bioresorbable implant composition of claim 1, the polymeric macro- or micro-scaffold comprising an alginate hydrogel.

11. The bioresorbable implant composition of claim 10, the alginate hydrogel being cross-linked with calcium sulfate.

12. The bioresorbable implant compositions of claim 1, the first bioactive agent comprising TGF-β, VEGF, and/or FGF-2 and the second bioactive agent comprising IGF-I and/or BMP-2.

13. The bioresorbable implant composition of claim 1, the first bioactive agent comprising a DNA plasmid encoding TGF-β, VEGF, and/or FGF-2 and the second bioactive agent comprising a DNA plasmid encoding IGF-I and/or BMP-2.

14. The bioresorbable implant composition of claim 2, the at least one cell comprising a progenitor cell.

15. A bioresorbable implant composition comprising:
a polymeric macro- or micro-scaffold; and
at least one bioactive agent incorporated on or within the polymeric macro- or micro-scaffold, the at least one bioactive agent modulating a function and/or characteristic of a cell.

16. The bioresorbable implant composition of claim 15 further including at least one cell dispersed on or within the polymeric macro- or micro-scaffold.

17. The bioresorbable implant composition of claim 16 further comprising at least one calcium phosphate nanoparticle dispersed on or within the polymeric macro- or micro-scaffold, the at least one calcium phosphate nanoparticle including at least one bioactive agent, the at least one calcium phosphate nanoparticle differentially releasing the at least one bioactive agent to modulate a function and/or characteristic of the at least one cell.

18. The bioresorbable implant composition of claim 17, the at least one calcium phosphate nanoparticle releasing the at least one bioactive agent with a different release profile than the release profile of a second bioactive agent from the same or different calcium phosphate nanoparticle.

19. The bioresorbable implant composition of claim 17, the at least one calcium phosphate nanoparticle degrading or diffusing before degradation or diffusion of a second calcium phosphate nanoparticle.

20. The bioresorbable implant composition of claim 15, the polymeric macro- or micro-scaffold comprising an alginate hydrogel.

21. The bioresorbable implant composition of claim 20, the alginate hydrogel being cross-linked with calcium sulfate.

22. The bioresorbable implant composition of claim 15, the at least one bioactive agent comprising at least one of TGF-β and IGF-I.

23. The bioresorbable implant composition of claim 15, the at least one bioactive agent comprising at least one of a DNA plasmid encoding FGF-2 and a DNA plasmid encoding BMP-2.

24. The bioresorbable implant composition of claim 16, the at least one cell comprising a progenitor cell.

25. A bioresorbable implant composition comprising:
a polymeric macro- or micro-scaffold; and
at least one interfering RNA molecule incorporated on or within the polymeric macro- or micro-scaffold, the at least one interfering RNA molecule modulating a function and/or characteristic of a cell.

26. The bioresorbable implant composition of claim 25 further including at least one cell dispersed on or within the polymeric macro- or micro-scaffold.

27. The bioresorbable implant composition of claim 26 further comprising at least one carrier material dispersed within the polymeric macro- or micro-scaffold, the at least carrier material differentially releasing the at least one interfering RNA molecule to modulate a function and/or characteristic of the at least one cell.

28. The bioresorbable implant composition of claim 26, the at least one carrier material releasing the at least one interfering RNA molecule with a different release profile than the release profile of a second interfering RNA molecule from the same or different carrier material.

29. The bioresorbable implant composition of claim 26, the at least one carrier material degrading or diffusing before degradation or diffusion of a second carrier material.

30. The bioresorbable implant composition of claim 26, the at least one carrier material comprising a degradable carrier material selected from the group consisting of calcium phosphate nanoparticles, PLGA microparticles, and combinations thereof.

31. The bioresorbable implant composition of claim 25, the polymeric macro- or micro-scaffold comprising an alginate hydrogel.

32. The bioresorbable implant composition of claim 31, the alginate hydrogel being cross-linked with calcium sulfate.

33. The bioresorbable implant composition of claim 25, the at least one interfering RNA molecule comprising an siRNA molecule.

34. The bioresorbable implant composition of claim 33, the siRNA molecule being capable of substantially silencing expression of a GNAS mRNA.

35. The bioresorbable implant composition of claim 26, the at least one cell comprising a progenitor cell.

36. The bioresorbable implant composition of claim 33, the siRNA molecule being capable substantially silencing expression of VEGF mRNA.
